(19) United States
(12) Patent Application Publication
(10) Pub. No.: US 2025/0352293 A1
(43) Pub. Date: Nov. 20, 2025

(54) MEDICAL APPARATUS AND CONTROL METHOD

(71) Applicant: CANON MEDICAL SYSTEMS CORPORATION, Tochigi (JP)

(72) Inventors: Takuya SAKAGUCHI, Utsunomiya (JP); Ryo HIRANO, Setagaya-ku (JP); Chihiro HATTORI, Nasushiobara (JP)

(73) Assignee: CANON MEDICAL SYSTEMS CORPORATION, Tochigi (JP)

(21) Appl. No.: 19/208,053

(22) Filed: May 14, 2025

(30) Foreign Application Priority Data
May 15, 2024 (JP) 2024-079747

(51) Int. Cl. A61B 90/35; G16H 40/67

(52) U.S. Cl. CPC A61B 90/35; G16H 40/67

(57) ABSTRACT

A medical apparatus according to an exemplary embodiment includes processing circuitry configured to acquire appearance information about a target area to be observed during a procedure in surgical treatment on a subject, and change a display state of a captured medical image of the target area based on the appearance information.

### CROSS-REFERENCE TO RELATED APPLICATIONS

This application is based upon and claims the benefit of priority from Japanese Patent Application No. 2024-079747, filed May 15, 2024, the entire contents of which are incorporated herein by reference.

### FIELD

Exemplary embodiments discussed in this specification and the accompanying drawings relate to medical apparatuses and control methods.

### BACKGROUND

In surgical treatment, doctors carefully observe medical images before surgery, create a treatment plan, and keep the planned images in mind before performing the procedure, conventionally. Therefore, various methods for displaying medical images observed before surgery have been proposed.

### BRIEF DESCRIPTION OF THE DRAWINGS

FIG. 1 is a block diagram illustrating an example of a configuration of a medical apparatus according to a first exemplary embodiment;

FIG. 2 is a flowchart illustrating an example of a processing procedure of the medical apparatus according to the first exemplary embodiment;

FIG. 3 is a diagram illustrating an example of change processing according to the first exemplary embodiment;

FIG. 4 is a diagram illustrating an example of change processing according to the first exemplary embodiment;

FIG. 5 is a diagram illustrating an example of display of a medical image according to the first exemplary embodiment;

FIG. 6 is a diagram illustrating an example of change processing according to the first exemplary embodiment;

FIG. 7 is a diagram illustrating an example of telesurgery according to the first exemplary embodiment.

FIG. 8 is a flowchart illustrating an example of a processing procedure of the medical apparatus according to a second exemplary embodiment;

FIG. 9 is a diagram illustrating an example of apparatus control according to the second exemplary embodiment;

FIG. 10 is a diagram illustrating an example of apparatus control according to the second exemplary embodiment;

FIG. 11 is a diagram illustrating an example of a display image according to a third exemplary embodiment.

### DETAILED DESCRIPTION

A medical apparatus according to an exemplary embodiment includes processing circuitry. The processing circuitry is configured to acquire appearance information about a target area to be observed during a procedure in surgical treatment on a subject. The processing circuitry is configured to change a display state of a captured medical image of the target area based on the appearance information.

Various Embodiments will be described hereinafter with reference to the accompanying drawings.

Hereinafter, exemplary embodiments of a medical apparatus and a control method will be described in detail with reference to the accompanying drawings. The medical apparatus and the control method according to the present application are not limited by the following exemplary embodiments. The following exemplary embodiments can be combined with another exemplary embodiment or the prior art without a contradiction arising in processing. In the following descriptions, similar components are assigned the same reference numerals, and the redundant description will be omitted.

In the medical apparatus and the control method according to the present exemplary embodiment, various types of processing are executed to cause the display state of a target area included in a medical image, to resemble the appearance of the target area to be observed during surgical treatment. Herein, the appearance of the target area to be observed during surgical treatment includes the appearance of the target area itself (actual object) to be observed during celiotomy, and the appearance of an endoscopic image to be observed during endoscopic surgery. The description of endoscopic surgery below also applies to laparoscopic surgery.

### First Exemplary Embodiment

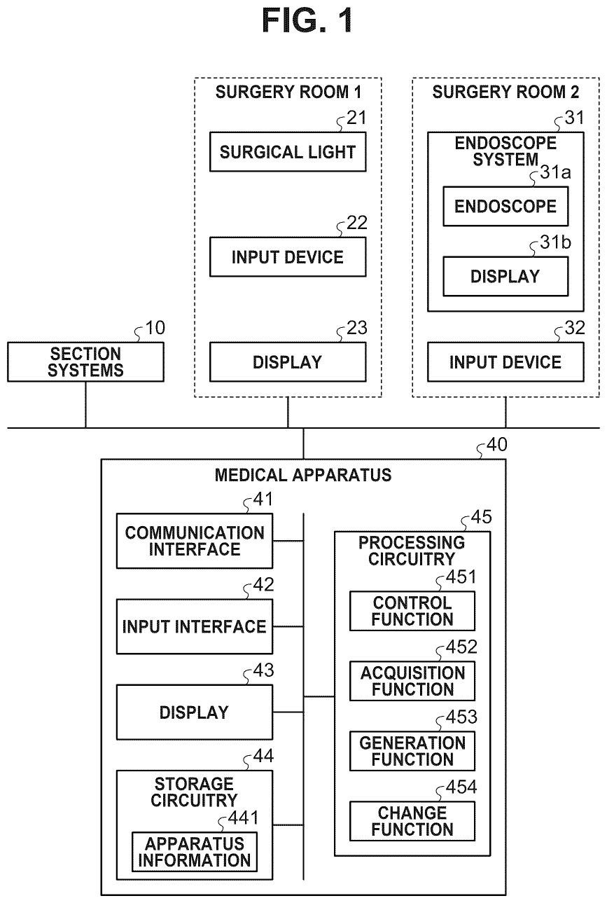

FIG. 1 is a block diagram illustrating an example of a configuration of a medical apparatus according to the first exemplary embodiment. For example, as illustrated in FIG. 1, a medical apparatus 40 is connected, via a network, to section systems, apparatuses installed in a surgery room 1, and apparatuses installed in a surgery room 2, in such a manner that communication can be performed with each other. The network includes, for example, an in-hospital local area network (LAN) or wide area network (WAN) installed in a hospital.

Other various apparatuses and systems such as medical image diagnostic apparatuses may be connected to the network illustrated in FIG. 1. The medical image diagnostic apparatus generates a medical image by capturing an image of a subject, and includes, for example, an X-ray computed tomography (CT) apparatus, a magnetic resonance imaging (MRI) apparatus, an X-ray diagnostic apparatus, an ultrasonic diagnostic, a single photon emission computed tomography (SPECT) apparatus, and a positron emission computed tomography (PET) apparatus.

While only the surgery room 1 and the surgery room 2 are illustrated in the network illustrated in FIG. 1, other surgery rooms may be further provided. In such a case, in the provided surgery room, the apparatuses connected to the medical apparatus 40 in such a manner that communication can be performed with each other is installed.

Section systems 10 include various systems such as a hospital information system (HIS), a radiology information system (RIS), a diagnosis report system, a laboratory information system (LIS), a rehabilitation section system, a dialysis section system, and a surgery section system. The medical apparatus 40 is connected to these systems, and transmits and receives various types of information to and from each other. For example, the medical apparatus 40 transmits and receives subject information, inspection information, treatment information, information regarding an analysis result, and information regarding a surgery (surgery information) to and from each system included in the section systems 10. For example, regarding a subject to be subjected to a surgery, the medical apparatus 40 acquires information regarding a surgery room to be used, the type of a surgery (celiotomy, endoscopic surgery, etc.), and information regarding an apparatus to be used for surgery, from the section systems 10.

The surgery room 1 is used, for example, for celiotomy and apparatuses such as a surgical light 21, an input device 22, and a display 23 are installed. In addition to these apparatuses and devices, a surgical table and a camera for capturing an image of a surgery, which are not illustrated in the drawings, are installed in the surgery room 1.

The surgical light 21 is an apparatus that illuminates a surgical field of the target area in a surgery in such a manner that an operator who performs the surgery can clearly view the surgical field. For example, the surgical light 21 includes a plurality of light source units, each of which illuminates the surgical field from different positions and directions, thus preventing shadows from forming in the surgical field. The surgical light 21 may include light source units each including a single light source (light bulb), or may include light source units each including a plurality of light sources. Examples of the light source include a light emitting diode (LED), an incandescent light bulb, a xenon tube, and an organic electro luminescence (EL).

For example, the surgical light 21 includes a control apparatus (not illustrated), and is configured to individually control a light emission amount and a wavelength of each light source of the respective light source units. The surgical light 21 is configured to optionally change a distance to the surgical table, an angle with respect to the surgical table, and a position with respect to the surgical table, using a drive mechanism to be driven by the above-described control apparatus. For example, the control apparatus operates based on control signals input by the input device 22, so that the surgical light 21 is controlled in terms of a light emission amount and a wavelength of each light source, the distance to the surgical table therefrom, the angle thereof with respect to the surgical table, and the position with respect to the surgical table. The surgical light 21 includes a grip portion, and a distance to the surgical table, the angle with respect to the surgical table, and the position with respect to the surgical table can also be manually changed by a manipulator gripping the grip portion.

The input device 22 inputs control information about the surgical light 21 to the medical apparatus 40, and control signals to the surgical light 21. Specifically, the input device 22 transmits, to the medical apparatus 40, control information including the light emission amount and the wavelength of each light source in the surgical light 21, the distance to the surgical table, the angle with respect to the surgical table, and the position with respect to the surgical table. The above-described control information is input by a manipulator via an input interface (not illustrated) included in the input device 22, for example. The input device 22 transmits, to the surgical light 21, a control signal that is based on the control information input from the manipulator via the input interface (not illustrated), and a control signal that is based on the control information received from the medical apparatus 40.

The display 23 displays various types of information and various types of data. For example, the display 23 displays a medical image in accordance with display control performed by the medical apparatus 40. For example, the display 23 displays medical images including a presurgical image, a treatment plan image, and a simulation image. The presurgical image is an image of a subject captured before a surgery. The treatment plan image is an image representing a treatment plan. The simulation image is an image on which various types of simulations have been performed. For example, the display 23 is implemented by a liquid crystal display, a cathode ray tube (CRT) display, or a touch panel.

The surgery room 2 is used for, for example, endoscopic surgery and apparatuses such as an endoscope system 31 and an input device 32 are installed. In addition to these apparatuses, a surgical table and a camera for capturing an image of a surgery, which are not illustrated, are installed in the surgery room 2. A display different from a display 31b of the endoscope system 31, which will be described below, may be installed in the surgery room 2.

The endoscope system 31 includes an endoscope 31a and the display 31b. The endoscope system 31 further includes processing circuitry that executes various types of processing and storage circuitry storing various types of information, which are not illustrated. The endoscope 31a includes an insertion section that is inserted into a subject, and an operation section for operating the insertion section. The insertion section includes a treatment section that performs treatment on a surgery target area (affected area) within a subject, and an imaging unit that captures an image inside the subject. The operation section receives operations performed by an operator on the treatment section and the imaging unit.

The treatment section includes, for example, forceps, an electrosurgical knife, and a suturing device. The imaging unit includes an image sensor, such as a charge coupled device (CCD) image sensor or a complementary metal oxide semiconductor (CMOS) image sensor, a lens, and a light emission unit, and captures an image of a target area to which light is emitted from the light emission units, using the image sensor.

The display 31b displays an image (endoscopic image) captured by the imaging unit. The processing circuitry (not illustrated) is connected to the endoscope 31a, the display 31b, and the storage circuitry (not illustrated), and controls the entire endoscope system 31. For example, the processing circuitry controls an operation of the treatment section in the endoscope 31a, collection of endoscopic images by the imaging unit, display of endoscopic images on the display 31b, and storing of endoscopic images in the storage circuitry. The storage circuitry stores endoscopic images collected by the imaging unit of the endoscope 31a.

The endoscope system 31 can also generate a virtual endoscopic image from medical images (e.g., computed tomography [CT] image, etc.) captured before surgery, and display the generated virtual endoscopic image on the display 31b in conjunction with an actual endoscopic image during the surgery. The endoscope system 31 can also display a medical image received from the medical apparatus 40, on the display 31b.

The input device 32 inputs control information about the endoscope system 31 to the medical apparatus 40, and control signals to the endoscope system 31. Specifically, the input device 32 transmits control information about the endoscope 31a in the endoscope system 31 to the medical apparatus 40. In such a case, for example, the input device 32 acquires the above-described control information from the endoscope system 31. The input device 32 transmits, to the endoscope system 31, the control signals that are based on the control information received from the medical apparatus 40.

As illustrated in FIG. 1, the medical apparatus 40 includes a communication interface 41, an input interface 42, a display 43, storage circuitry 44, and processing circuitry 45. For example, the medical apparatus 40 is implemented by a computer device, such as a server or a work station. The medical apparatus 40 according to the first exemplary embodiment acquires appearance information about a target area under surgical treatment based on apparatus information about an apparatus installed in the corresponding surgery room, and changes a display state of a captured medical image of the target area based on the acquired appearance information.

Specifically, the medical apparatus 40 according to the first exemplary embodiment changes the display state of a medical image in such a manner as to resemble the appearance of the target area under a surgical treatment, both when a presurgical medical image is observed and when an intraoperative medical image is observed. For example, when a presurgical medical image is observed (e.g., a timing at which a treatment plan is formulated with reference to a medical image, a timing at which surgery simulation is performed, etc.), the medical apparatus 40 estimates the appearance of a target area expected to be observed during surgery, based on surgery information and information regarding an apparatus installed in a surgery room, and changes the display state of the medical image in such a manner as to become a display state resembling the estimated appearance. In observation of an intraoperative medical image, the medical apparatus 40 estimates the appearance of a target area being observed during surgery, based on control information about an apparatus under the surgery as well as information regarding the apparatus, and changes the display state of the medical image to a display state resembling the estimated appearance.

The communication interface 41 controls the transmission and communication of various types of data to be communicated between the medical apparatus 40 and each apparatus connected via the network. Specifically, the communication interface 41 is connected to the processing circuitry 45, and transmits data received from each apparatus on the network, to the processing circuitry 45, or transmits data received from the processing circuitry 45, to each apparatus on the network. For example, the communication interface 41 is implemented by a network card, a network adapter, or a network interface controller (NIC).

The input interface 42 receives input operations for various instructions and various types of information from a manipulator. Specifically, the input interface 42 is connected to the processing circuitry 45, converts an input operation received from the manipulator, into an electric signal, and transmits the electric signal to the processing circuitry 45. For example, the input interface 42 is implemented by a trackball, a switch button, a mouse, a keyboard, a touch-pad with which an input operation is performed with a touch on an operation surface, a touch screen with a display screen and a touch-pad integrated, a contactless input interface that uses an optical sensor, and a voice input interface. In this specification, the input interface 42 is not limited to those that include physical operation components, such as a mouse and a keyboard. For example, electric signal processing circuitry that receives an electric signal corresponding to an input operation, from an external input device provided separately from an apparatus, and transmits the electric signal to control circuitry is also included as an example of the input interface 42. The input interface is an example of a reception unit.

The display 43 displays various types of information and various types of data. Specifically, the display 43 is connected to the processing circuitry 45, and displays various types of information and various types of data (e.g., medical image) received from the processing circuitry 45. For example, the display 43 is implemented by a liquid crystal display, a cathode ray tube (CRT) display, or a touch panel.

The storage circuitry 44 stores various types of data and various programs. Specifically, the storage circuitry 44 is connected to the processing circuitry 45, and stores data received from the processing circuitry 45, or reads stored data and transmits the data to the processing circuitry 45. For example, the storage circuitry 44 is implemented by a semiconductor memory device, such as a read only memory (ROM), a random access memory (RAM), or a flash memory, a hard disc, or an optical disc. For example, the storage circuitry 44 stores medical images collected from a subject before surgery. As illustrated in FIG. 1, the storage circuitry 44 stores apparatus information 441. The apparatus information 441 is information regarding an apparatus for the respective surgery rooms. For example, the storage circuitry 44 stores, as the apparatus information 441, information (manufacturer name, model number, catalog information, etc.) regarding the surgical light 21 installed in the surgery room 1, and information (manufacturer name, model number, catalog information, etc.) regarding the endoscope system 31 installed in the surgery room 2. The storage circuitry 44 may be implemented by a cloud computer connected to the medical apparatus 40 via the network.

The processing circuitry 45 controls the entire medical apparatus 40. Specifically, the processing circuitry 45 controls communication of information with each apparatus on the network, and controls various types of processing regarding surgical treatment. The processing circuitry 45 can also perform various types of processing in accordance with an input operation performed via the input interface 42.

The configuration example of the medical apparatus 40 according to the present exemplary embodiment has been described above. For example, the medical apparatus 40 is installed in a medical care facility such as a hospital, and supports various types of diagnosis and treatment plan formulation that are to be performed by a user such as a doctor. The medical apparatus 40 executes various types of control in surgical treatment. Hereinafter, the medical apparatus 40 having such a configuration will be described in detail.

For example, as illustrated in FIG. 1, the processing circuitry 45 of the medical apparatus 40 executes a control function 451, an acquisition function 452, a generation function 453, and a change function 454. The control function 451 is an example of a display control unit. The acquisition function 452 is an example of an acquisition unit. The change function 454 is an example of a change unit.

The control function 451 controls the display of various types of information on the display 43 and the display 23 installed in the surgery room 1. The control function 451 controls the surgical light 21 by transmitting control information to the input device 22 installed in the surgery room 1. The control function 451 controls the endoscope system 31 by transmitting control information to the input device 32 installed in the surgery room 2.

The acquisition function 452 acquires various types of information from the section systems 10. For example, the acquisition function 452 acquires, from the section systems 10, medical images collected in advance from a subject to be subjected to a surgical operation. The acquisition function 452 can acquire, as the above-described medical image, presurgical images (raw data collected by a medical image diagnosis apparatus, image data obtained after reconstruction of the raw data, images obtained by executing various types of image processing on image data), a treatment plan image, and a simulation image.

The acquisition function 452 acquires appearance information about a target area to be observed during a procedure in surgical treatment on a subject. Specifically, the acquisition function 452 acquires surgery information about the subject to be subjected to surgical treatment, and acquires appearance information indicating the appearance of the target area in surgery, based on the surgery information. More specifically, the acquisition function 452 acquires, from the section systems 10, information regarding the surgery room where the surgery is to be performed on the subject, and information regarding the apparatus(es) to be used in the surgery. The acquisition function 452 refers to the apparatus information 441 stored in the storage circuitry 44, reads apparatus information corresponding to the acquired information, and acquires appearance information about the target area in the surgery based on the read apparatus information. The processing to be performed by the acquisition function 452 will be described in detail below.

The generation function 453 generates various images based on medical images acquired by the acquisition function 452. For example, the generation function 453 generates image data by performing known reconstruction processing on raw data. The generation function 453 generates various display images by performing known image processing (e.g., rendering processing, etc.) on image data. The generation function 453 can also generate a treatment plan image and a simulation image in accordance with an input operation performed by a manipulator via the input interface 42.

The change function 454 changes the display state of a captured medical image of a target area based on appearance information. Specifically, the change function 454 changes the display state of the medical image acquired by the acquisition function 452, based on the appearance information about the target area acquired by the acquisition function 452. More specifically, the change function 454 changes the display state of the medical image in such a manner as to resemble the appearance of the target area corresponding to the time of surgery. For example, the change function 454 changes at least one of contrast and color as the display state of the medical image. The change function 454 may change the display state of the medical image by changing display condition(s) of the medical image acquired by the acquisition function 452, or may change the display state of the medical image by causing the generation function 453 to generate a new medical image using the medical image acquired by the acquisition function 452. The processing to be performed by the change function 454 will be described in detail below.

The above-described processing circuitry 45 is implemented by a processor, for example. In this case, each of the above-described processing functions is stored in the storage circuitry 44 in the form of a program executable by a computer. The processing circuitry 45 implements a function corresponding to the respective programs, by reading programs stored in the storage circuitry 44 and executing the read programs. In other words, the processing circuitry 45 has the processing functions illustrated in FIG. 1, in a state in which a corresponding program is read out.

The processing circuitry 45 may include a plurality of independent processors, and each processor may implement a corresponding processing function by executing a program. The processing functions of the processing circuitry 45 may be implemented with being appropriately distributed to or integrated into a single piece of or a plurality of pieces of processing circuitry. The processing functions of the processing circuitry 45 may be implemented by the mixture of hardware such as circuitry or software. Here, a description has been provided of an example case where programs corresponding to processing functions are stored in a single piece of storage circuitry 44, but the exemplary embodiment is not limited to this. For example, programs corresponding to processing functions may be stored with being distributed to a plurality of pieces of storage circuitry, and the processing circuitry 45 may read out and execute a program from each piece of storage circuitry. A part of the processing functions of the processing circuitry 45 may be implemented by a cloud computer connected to the medical apparatus 40 via a network.

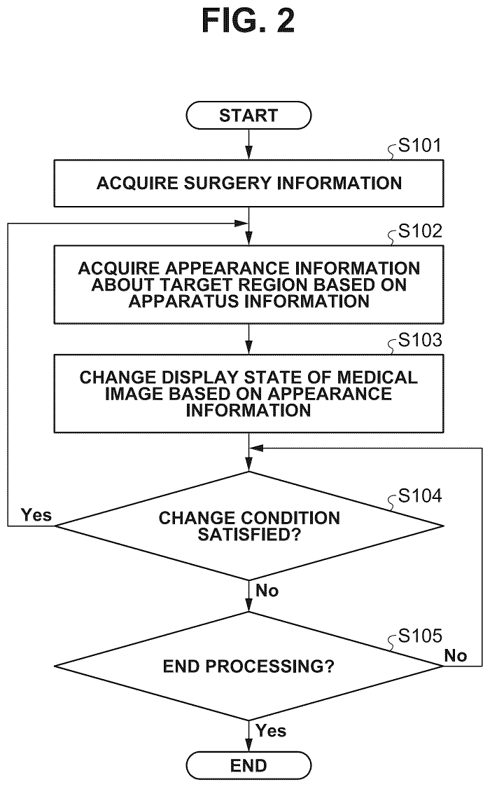

Next, a procedure of processing to be performed by the medical apparatus 40 according to the first exemplary embodiment will be described with reference to FIG. 2, and then details of each piece of processing will be described. FIG. 2 is a flowchart illustrating an example of a processing procedure of the medical apparatus 40 according to the first exemplary embodiment.

For example, in the present exemplary embodiment, in step S101, the acquisition function 452 acquires surgery information about a subject who is to undergo surgical treatment, and in step S102, acquires appearance information about a target area based on apparatus information corresponding to the acquired surgery information, as illustrated in FIG. 2. This process is implemented by, for example, the processing circuitry 45 calling a program corresponding to the acquisition function 452, from the storage circuitry 44, and executing the program.

Subsequently, in step S103, the change function 454 changes the display state of the medical image based on the appearance information. Specifically, the change function 454 controls the generation function 453 and/or the control function 451 to display the medical image resembling the appearance of the target area corresponding to the time of surgery. In step S104, the change function 454 determines whether a change condition for changing the display state of the medical image is satisfied. The processing is implemented by, for example, the processing circuitry 45 calling programs corresponding to the change function 454, the control function 451, and the generation function 453, from the storage circuitry 44, and executing the programs.

In step S104, if the change function 454 determines that the change condition is satisfied (YES in step S104), the processing returns to step S102, in which the acquisition function 452 acquires appearance information. If the change function 454 determines in step S104 that the change condition is not satisfied (NO in step S104), the processing proceeds to step S105. In step S105, the change function 454 determines whether to end the processing. If the change function 454 determines that the processing is not to be ended (NO in step S105), the processing returns to step S104. In step S104, the change function 454 continues the determination in step S104. If the change function 454 determines that the processing is to be ended (YES in step S105), the medical apparatus 40 ends the processing. The processing is implemented by, for example, the processing circuitry 45 calling a program corresponding to the change function 454, from the storage circuitry 44, and executing the program.

Hereinafter, details of processes to be performed by the medical apparatus 40 will be described. The medical apparatus 40 according to the first exemplary embodiment executes a different type of processing in accordance with the type of surgery to be performed. Specifically, processing to be performed by the medical apparatus 40 varies between a case where celiotomy is performed as surgical treatment, and a case where endoscopic surgery is performed. Hereinafter, the processing to be performed during celiotomy and the processing to be performed during endoscopic surgery will be described in order.

### (Processing During Celiotomy)

Hereinafter, processing to be performed during celiotomy will be described along the processing described in conjunction with the flowchart in FIG. 2.

### (Acquisition Processing of Surgery Information)

As described with reference to step S101 of FIG. 2, the acquisition function 452 acquires surgery information about a subject on which surgical treatment is performed. Specifically, the acquisition function 452 acquires the type of surgical treatment that a subject undergoes, information regarding a surgery room where the surgical treatment is to be performed, and information regarding an apparatus to be used in the surgical treatment. For example, the acquisition function 452 acquires surgery information about a subject from the section systems 10 based on subject information input via the input interface 42. For example, the acquisition function 452 acquires information indicating that celiotomy is to be performed in the surgery room 1, as surgery information about a subject. The above-described surgery information may be acquired by being input simultaneously with the input of subject information via the input interface 42.

### (Acquisition Processing of Appearance Information)

As described in conjunction with step S102 of FIG. 2, the acquisition function 452 acquires appearance information about the target area of surgical treatment based on apparatus information corresponding to the surgery information acquired in step S101. Specifically, the acquisition function 452 initially acquires apparatus information corresponding to surgery information, from pieces of the apparatus information 441 stored in the storage circuitry 44. For example, the acquisition function 452 acquires, as the apparatus information corresponding to the surgery room 1 that has been acquired as surgery information, information (manufacturer name, model number, catalog information, etc.) regarding the surgical light 21.

The acquisition function 452 acquires appearance information indicating the appearance of the target area during surgery, from the acquired apparatus information (information regarding the surgical light 21). Specifically, the acquisition function 452 acquires appearance information about the target area that is based on illumination conditions of a lighting apparatus (the surgical light 21) to be used during a procedure in surgical treatment.

In the medical apparatus 40, various methods can be used when the appearance information about the target area is acquired from apparatus information. For example, information indicating the appearance of the target area may be preliminarily acquired for each lighting apparatus, and stored in association with the apparatus information 441 in the storage circuitry 44, and the associated appearance information may be acquired together when the acquisition function 452 acquires apparatus information. In this case, for example, the appearance information may be information indicting, for each type of target area (procedure of celiotomy), the appearance (contrast or color of the target area) of the target area (target area in a state of being illuminated with light after abdominal opening) under various illumination conditions including the position and the angle of the surgical light 21 with respect to the surgical table, a light emission amount, and a wavelength of the surgical light 21.

From the appearance information associated with apparatus information, the acquisition function 452 acquires appearance information that corresponds to the target area of surgical treatment and corresponds to the illumination conditions of the surgical light 21 at the present time. For example, at the time of a presurgical treatment planning or simulation, the acquisition function 452 acquires the appearance information corresponding to the illumination conditions set by default, and acquires the corresponding appearance information each time any illumination condition is changed (condition is changed via the input interface 42). In other words, the acquisition function 452 acquires the appearance information corresponding to the illumination conditions suitable for a planned procedure, and acquires the corresponding appearance information even in a case where an illumination condition is changed.

For example, during surgery, the acquisition function 452 constantly acquires the illumination conditions of the surgical light 21 at the present time, and acquires the appearance information corresponding to the acquired illumination conditions. The acquisition function 452 acquires appearance information about the target area that is based on the illumination conditions of a lighting apparatus used during a procedure. Further, the acquisition function 452 acquires appearance information about the target area that is based on the illumination conditions of the lighting apparatus for each progress status of a procedure. In other words, the acquisition function 452 acquires the appearance information about the target area each time new control information is received from the input device 22.

The medical apparatus 40 can also use a three-dimensional model as a method of acquiring appearance information about the target area from apparatus information. In this case, for example, a simulation model in which three-dimensional models of a surgical table, the surgical light 21, and a subject are arranged in a virtual space that simulates the surgery room 1 is preliminarily generated, and stored in the storage circuitry 44. The acquisition function 452 acquires appearance information about the target area by reading the simulation model and illuminating the three-dimensional model of the surgical light 21 based on the illumination conditions at the present time. The positional relationship between the respective area in the three-dimensional model of the subject and the corresponding area in a medical image can be correlated based on, for example, anatomical feature points through alignment. As a three-dimensional model of a subject, a three-dimensional medical image collected from the subject may be used.

In a case where the above-described simulation model is used, the acquisition function 452 can acquire the appearance information for each illumination condition by changing illumination of the three-dimensional model of the surgical light 21 each time any of the illumination conditions is changed. For example, at the time of presurgical treatment planning or simulation, or during surgery, the acquisition function 452 can acquire appearance information each time an illumination condition is changed (new control information is received from the input device 22). In the above-described simulation model, the abdominal opening of a subject can also be simulated. The acquisition function 452 can acquire the appearance information about the target area in accordance with a state of the abdominal opening of the subject.

### (Change Processing of Display State)

As described in conjunction with step S103 of FIG. 2, the change function 454 changes the display state of the medical image based on appearance information acquired by the acquisition function 452. Specifically, the change function 454 changes the display state of the medical image in such a manner that the display state of the target area in the medical image resembles the appearance of the target area that is indicated by the appearance information.

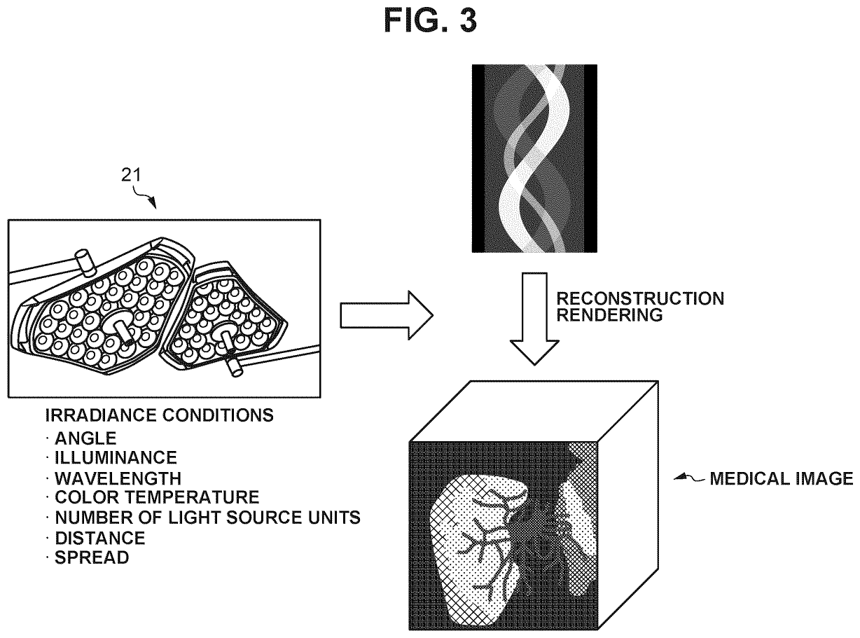

FIG. 3 is a diagram illustrating an example of change processing according to the first exemplary embodiment. For example, as illustrated in FIG. 3, the change function 454 changes the display state of the medical image in such a manner as to resemble the appearance of the target area illuminated by the surgical light 21. The illumination conditions of the surgical light 21 include, for example, the illumination angle of each light source unit, illuminance of each light source unit, wavelength of each light source unit, and color temperature of each light source unit, the number of light source units, a distance from each light source unit to the target area, and light spread from each light source unit. The appearance information (information regarding contrast and color of the target area) corresponding to the illumination conditions at the present time is acquired by the acquisition function 452.

The change function 454 changes the display state of the medical image of the target area in such a manner as to resemble the contrast and the color of the target area that are indicated by the appearance information. For example, the change function 454 controls reconstruction processing and rendering processing of a medical image in such a manner that a bright portion or a dark portion (or shadowed portion) in the target area appears similar to the appearance indicated by the appearance information. The change function 454 controls the generation function 453 to generate a medical image in a state similar to the state indicated by the appearance information.

For example, the change function 454 controls the color of a medical image in such a manner that the color of each portion in the target area appears similar to the color indicated by the appearance information. The change function 454 controls the control function 451, thus controlling the color of each portion in a medical image that is to have a color similar to the color indicated by the appearance information.

The information regarding the color of the target area may be acquired by the camera installed in the surgery room 1. In this case, the change function 454 identifies each area in a surgical field from an image acquired by the camera. The area identification may be performed through image analysis or a known method that uses artificial intelligence (AI). The change function 454 acquires information regarding the color of the identified area, from the image, and changes the display state of the medical image by reflecting the acquired color information in the corresponding area in the medical image.

For example, the change function 454 controls the display states of various medical images such as a presurgical image, a treatment plan image, and a simulation image to resemble the appearance of the target area that is indicated by the appearance information. The control function 451 controls a medical image of which the display state has been changed by the change function 454, to be displayed on the display 43 and the display 23. For example, in the case of a medical image to be observed at the time of presurgical treatment planning or simulation, the control function 451 displays the medical image of which the display state has been changed by the change function 454, on the display 43. For example, in the case of a medical image to be observed during surgery, the control function 451 displays the medical image of which the display state has been changed by the change function 454, on the display 23.

### (Change Determination Processing)

As described in conjunction with step S104 of FIG. 2, the change function 454 determines whether a change condition is satisfied. Specifically, the change function 454 determines whether any illumination condition of the lighting apparatus has been changed. For example, during surgery, illumination condition(s), such as the position, the angle, and a light emission amount of the surgical light 21, is/are sometimes changed in accordance with the progress of a procedure. In response to the change function 454 detecting a change in an illumination condition, the acquisition function 452 acquires the appearance information corresponding to the changed illumination condition. The acquisition function 452 acquires appearance information about the target area that is based on an illumination condition of a lighting apparatus in each progress status of a procedure. Each time appearance information is acquired, the change function 454 changes the display state of the medical image based on the acquired appearance information.

### (Operation of Determining End of Processing)

As described in conjunction with step S105 of FIG. 2, the change function 454 determines whether to end the processing of changing the display state of a medical image. For example, in a case where the display state of a medical image observed before surgery is changed, the change function 454 determines whether the display of the medical image on the display 43 has been ended, thus determining whether to end the processing. For example, in a case where the display state of a medical image observed during surgery has been changed, the change function 454 determines whether the display of the medical image on the display 23 has been ended, thus determining whether to end the processing.

In the above-described exemplary embodiment, a case where an illumination condition is changed in accordance with the type of target area or a progress status of a procedure has been described. The exemplary embodiment is not limited to this. For example, illumination condition(s) of the lighting apparatus (e.g., the surgical light 21) may be changed in accordance with the number of doctors who perform surgery and/or their standing positions. For example, in a case where a plurality of doctors performs surgery, the illumination conditions of the surgical light 21 are determined based on the standing position of each doctor, so that no shadow is formed in the target area. The acquisition function 452 acquires the appearance information about the target area based on the determined illumination condition. The standing position of each doctor is predetermined, and, for example, a three-dimensional model of each doctor is arranged at the corresponding position in the above-described simulation model, so that the illumination conditions for the target area is determined.

### (Processing to be Performed During Endoscopic Surgery)

Next, processing to be performed during endoscopic surgery will be described along the processing described in conjunction with the flowchart in FIG. 2.

### (Acquisition Processing of Surgery Information)

In the processing to be performed during endoscopic surgery, the acquisition function 452 acquires, as surgery information about a subject, information indicating that the endoscopic surgery is to be performed in the surgery room 2, for example.

### (Processing of Acquiring Appearance Information)

In the processing to be performed during endoscopic surgery, the acquisition function 452 acquires, as the apparatus information corresponding to the surgery room 2 acquired as the surgery information, information (manufacturer name, model number, catalog information, etc.) regarding the endoscope system 31. Specifically, the acquisition function 452 acquires information regarding the insertion section (treatment section and imaging unit) in the endoscope 31a of the endoscope system 31. For example, the acquisition function 452 acquires information such as the shape of the insertion section of the endoscope 31a, the number and position of light emission units, and a light emission amount, a wavelength, and color of emitted light.

The acquisition function 452 acquires, from the acquired apparatus information (information regarding the insertion section of the endoscope 31a), appearance information indicating the appearance of the target area during endoscopic surgery. The acquisition function 452 acquires different appearance information between a case where a medical image is observed before surgery, and a case where a medical image is observed during surgery.

For example, in the observation of a presurgical medical image, the acquisition function 452 acquires the appearance information about the target area that is based on the illumination conditions of the light emission unit in the endoscope 31a. In the observation of a presurgical medical image, the acquisition function 452 acquires the appearance information about the target area based on the illumination conditions of light, as in celiotomy.

For example, as in celiotomy, information indicating the appearance of the target area may be preliminarily acquired for each endoscope 31a, and stored in association with the apparatus information 441 of the storage circuitry 44, and the associated appearance information may be acquired together when the acquisition function 452 acquires the apparatus information.

From the pieces of the appearance information associated with the apparatus information, the acquisition function 452 acquires the appearance information that corresponds to the target area of the surgical treatment and corresponds to the illumination conditions of the endoscope 31a at the present time. For example, at the time of a presurgical treatment planning or simulation, the acquisition function 452 acquires the appearance information corresponding to the illumination conditions set by default, and acquires the corresponding appearance information each time any of the illumination conditions is changed (condition is changed via the input interface 42). The acquisition function 452 acquires the appearance information corresponding to the illumination conditions suitable for a planned procedure, and acquires the corresponding appearance information even in a case where an illumination condition is changed.

Also during endoscopic surgery, appearance information can be acquired using a three-dimensional model. In this case, for example, a simulation model in which a three-dimensional model of a surgical table, a three-dimensional model of the endoscope 31a, and a three-dimensional model of a subject are arranged in a virtual space simulating the surgery room 2 is preliminarily generated, and stored in the storage circuitry 44. The acquisition function 452 acquires the appearance information about the target area by reading out the simulation model and illuminating a light emission unit in the three-dimensional model of the endoscope 31a based on the illumination conditions at the present time (the position and orientation of the insertion section in the subject, and a light emission amount, a wavelength, and color of light emitted from the light emission unit, etc.). As a positional relationship between each area in the three-dimensional model of the subject and the corresponding area in a medical image, the areas can be associated through alignment based on anatomical feature points, for example. As a three-dimensional model of a subject, a three-dimensional medical image collected from the subject may be used.

Next, in the observation of an intraoperative medical image, the acquisition function 452 acquires an endoscopic image of the target area that is captured by the endoscope 31a, as appearance information. Specifically, the acquisition function 452 acquires the appearance information about the target area in a captured image that is based on the display condition of the captured image (endoscopic image) of the target area that has been captured during a procedure of surgical treatment. For example, the acquisition function 452 acquires, as the appearance information, the state (contrast, color, etc. of each portion) of the target area depicted in the endoscopic image.

An area in an endoscopic image and the corresponding area in a medical image can be associated through alignment based on anatomical feature points, for example. For example, the acquisition function 452 estimates an area depicted in an endoscopic image, from anatomical feature points included in the endoscopic image, and identifies the corresponding position in the medical image based on the estimated area.

The association between an area in an endoscopic image and the corresponding area in a medical image may be performed using a position sensor. In such a case, the position sensor acquires the position and the orientation of the insertion section of the endoscope 31a in the subject, so that an area depicted in the currently-captured endoscopic image is estimated, and the corresponding position in the medical image is identified based on the estimated area. For example, the position and the orientation of the insertion section of the endoscope 31a in the above-described simulation model are set to the position and the orientation acquired by the position sensor, so that the acquisition function 452 estimates the area depicted in the currently-captured endoscopic image. The acquisition function 452 then identifies the corresponding position in the medical image based on the estimated area.

### (Display State Change Processing)

Also in the processing to be performed during endoscopic surgery, the change function 454 changes the display state of a medical image in such a manner that the display state of the target area in the medical image resembles the appearance of the target area that is indicated by the appearance information.

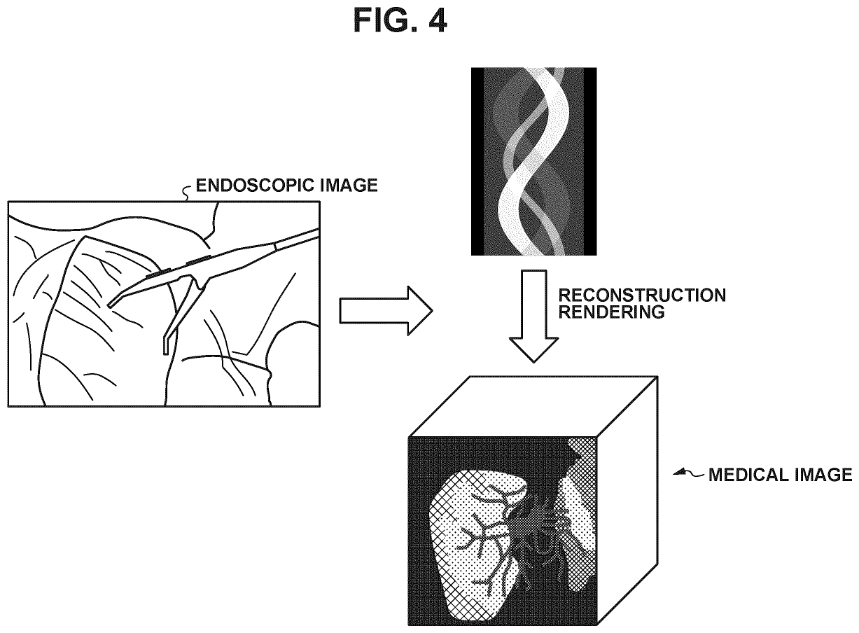

FIG. 4 is a diagram illustrating an example of change processing according to the first exemplary embodiment. FIG. 4 illustrates an example case where an endoscopic image is acquired as appearance information. For example, as illustrated in FIG. 4, the change function 454 changes the display state of a medical image of the target area to a state resembling the corresponding target area in the endoscopic image in terms of the contrast and color thereof. For example, the change function 454 controls reconstruction processing and rendering processing of a medical image in such a manner that a bright portion or a dark portion (or shadowed portion) in the target area appear similar to those in the endoscopic image. The change function 454 controls the generation function 453, thus generating a medical image reflecting a state similar to the state represented by the appearance information.

For example, the change function 454 controls the colors of a medical image in such a manner that the colors of each portion in the target area appears similar to the colors indicated by the appearance information. The change function 454 controls the colors of each portion in the medical image that is to be made similar to those indicated by the appearance information under the control of the control function 451. For example, the change function 454 changes the display state in such a manner that an organ in which blood circulates is indicated in red, and an organ in an anemic state is indicated in blue.

For example, the change function 454 controls the display states of various medical images, such as a presurgical image, a treatment plan image, and a simulation image, to resemble the appearance of the target area that is indicated by the appearance information. The control function 451 controls a medical image of which the display state has been changed by the change function 454, to be displayed on the display 43 and the display 31b of the endoscope system 31. For example, in the case of a medical image to be observed at the time of presurgical treatment planning or simulation, the control function 451 displays a medical image of which the display state has been changed by the change function 454, on the display 43.

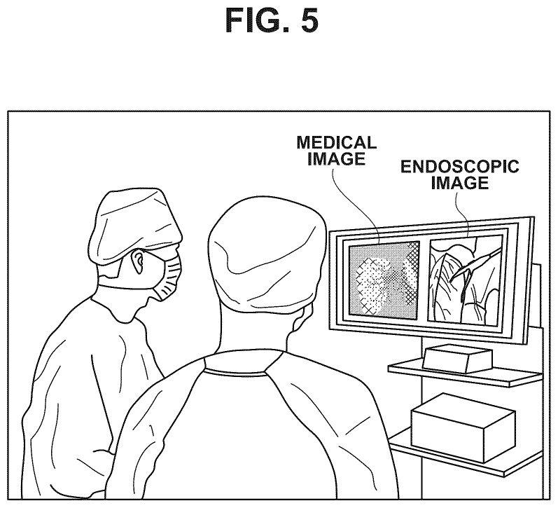

For example, in the case of a medical image to be observed during surgery, the control function 451 performs control to display the medical image of which the display state has been changed by the change function 454, on the display 31b. By transmitting the medical image of which the display state has been changed, to the endoscope system 31, the control function 451 displays the medical image on the display 31b in the endoscope system 31 as illustrated in FIG. 5. A doctor can thereby observe an endoscopic image and a medical image that are arranged side by side, during surgery. FIG. 5 is a diagram illustrating an example of display of a medical image according to the first exemplary embodiment.

In an endoscope system or a system that uses a surgery support robot, a system that displays a three-dimensional endoscopic image in which the target area can be stereoscopically viewed has been known. The medical apparatus 40 according to the present exemplary embodiment can also acquire a stereoscopically-viewable three-dimensional endoscopic image in such a system as appearance information, and change the display state of a medical image.

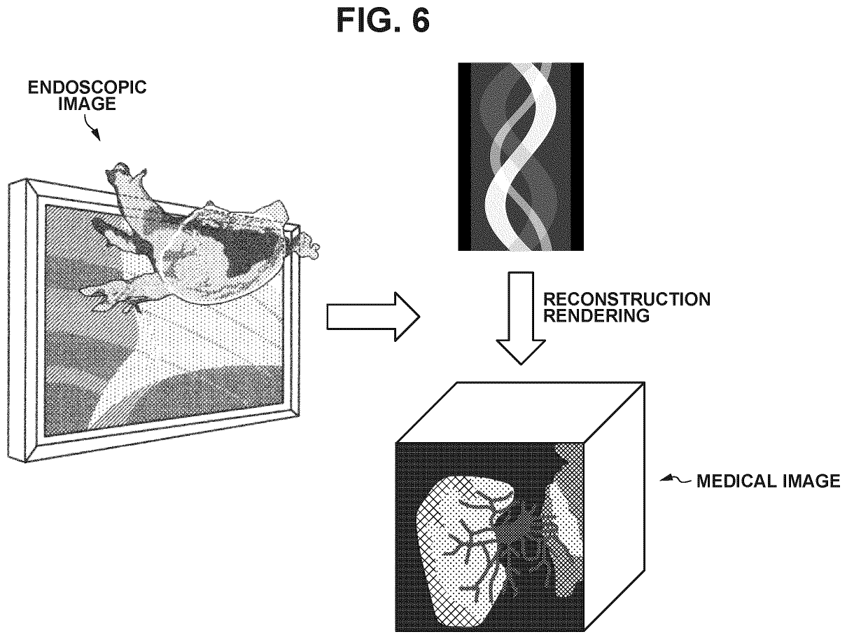

FIG. 6 is a diagram illustrating an example of change processing according to the first exemplary embodiment. As illustrated in FIG. 6, in the case of displaying a medical image similar to the target area to be stereoscopically viewed in an endoscopic image, the acquisition function 452 acquires appearance information including a portion to be shadowed in a stereoscopic view, for example. The change function 454 changes the display state of the medical image in such a manner as to shadow the portion to be shadowed in a stereoscopic view, based on the acquired appearance information.

### (Change Determination Processing)

In the processing to be performed during endoscopic surgery, the change function 454 determines whether a change condition is satisfied, by determining whether a condition of the endoscope 31a has changed. For example, during surgery, condition(s), such as the position and the angle of the insertion section of the endoscope 31a, and a light emission amount of a light emission unit, is/are sometimes changed in accordance with the progress of a procedure. If a change in such a condition is detected by the change function 454, the acquisition function 452 acquires the appearance information corresponding to the changed condition.

### (Processing End Determination Processing)

Also in the processing to be performed during endoscopic surgery, the change function 454 determines whether to end the processing, by determining whether the display of a medical image on the display 43 or the display 31b has been ended.

### Modified Example 1

In the above-described exemplary embodiment, a description has been provided of a case where a single medical image is displayed, and the display state of the displayed medical image is changed in accordance with the appearance of the target area. The exemplary embodiment is not limited to this, and a plurality of medical images may be displayed. In this case, a display condition for each medical image is changed in such a manner that a plurality of medical images resembles the appearance of the target area. Among a plurality of medical images, the display state of only limited medical image(s) may be changed. For example, among a plurality of medical images, the display state of only a medical image to be observed by a doctor who mainly performs surgery may be changed.

The medical apparatus 40 can also display medical images that appear differently to a plurality of doctors. For example, in a case where a plurality of doctors performs surgery, the appearance of the target area varies depending on the position with respect to a subject (patient). For this reason, the medical apparatus 40 changes the display states of medical images in accordance with the appearance of the target area to be observed by the respective doctors. For example, in a case where the medical apparatus 40 displays medical images to a doctor A who is planned to stand on the right side of a subject and a doctor B who is planned to stand on the left side during a surgery, the medical apparatus 40 acquires appearance information about the target area to be observed from the standing position of the respective doctors, and changes the display states of the medical images in accordance with the acquired appearance information.

In this case, for example, the acquisition function 452 reads the above-described simulation model, illuminates the three-dimensional model of the surgical light 21 based on an illumination condition at the present time, and acquires appearance information about the target area to be observed from the standing position of the doctor A (the orientation of the target area, a positional relationship of the target area with other organs, the contrast of light, and the color, etc.), and appearance information about the target area to be observed from the standing position of the doctor B (the orientation of the target area, the positional relationship of the target area with other organs, the contrast of light, and the color, etc.).

The change function 454 changes the display state of the medical images by changing the appearance information to be used, between a case where the doctor A observes the medical image and a case where the doctor B observes the medical image. In other words, for the medical image to be observed by the doctor A, the change function 454 changes the display state of the medical image (the orientation of the target area, the positional relationship of the target area with other organs, the contrast of light, and the color, etc.) in such a manner as to resemble the appearance indicated by the appearance information about the target area that can been seen in a case where the target area is observed from the right side of the subject. In contrast, for the medical image to be observed by the doctor B, the change function 454 changes the display state of the medical image (the orientation of the target area, the positional relationship of the target area with other organs, the contrast of light, and the color, etc.) in such a manner as to resemble the appearance indicated by the appearance information about the target area that can be seen in a case where the target area is observed from the left side of the subject.

As described above, by changing the display state of a medical image to be displayed, in accordance with a standing position in surgery, the medical apparatus 40 can display an appropriate medical image for each doctor who performs surgery. For example, in image training that uses a presurgical plan image, it is possible for individual doctors to observe a medical image resembling the appearance of the target area to be observed, and perform appropriate image training. Further, regarding the medical image to be observed during a surgery, it is possible to display medical images to be observed, in a display state appropriate for the respective doctors, thus realizing appropriate support during surgery.

### Modified Example 2

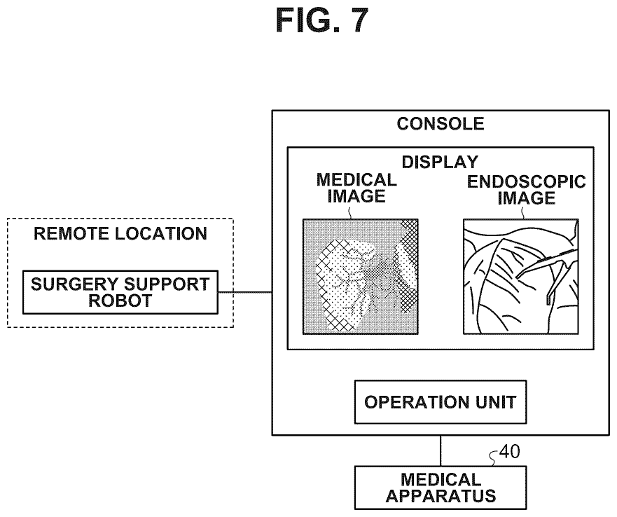

In the above-described exemplary embodiment, a case where a doctor who performs surgery performs surgery in the surgery room 1 or the surgery room 2 has been described. The exemplary embodiment is not limited to this. For example, the exemplary embodiment is applicable to a case where telesurgery is performed on a subject in a remote location. FIG. 7 is a diagram illustrating an example of a telesurgery according to the first exemplary embodiment. As illustrated in FIG. 7, for example, in telesurgery, a surgery support robot is installed in a remote location where a subject is present, and a console for operating the surgery support robot is installed in a hospital where a surgeon is present.

The surgeon operates a robot arm of the surgery support robot by operating an operation section while observing an endoscopic image displayed on a display in the console. The robot arm holds medical instruments such as forceps and an endoscope, and the surgeon performs surgery by operating the operation section to operate these medical instruments.

In such telesurgery, the medical apparatus 40 can display a medical image similar to an endoscopic image on the display of the console. In telesurgery, in addition to a case where a medical image similar to an endoscopic image is displayed, a medical image that can be easily observed by a surgeon may be displayed. In other words, in a case where surgical treatment is performed through a remote manipulation, the change function 454 can change the display state of a medical image in accordance with the manipulator of a remote manipulation who observes a captured image (endoscopic image) of the target area.

In this case, the medical apparatus 40 preliminarily receives information regarding color and shadow states desired by the surgeon, and stores the received information. When telesurgery is performed, the change function 454 reads out information in accordance with a display state change instruction from the surgeon, and controls the display state of a medical image to change the display state to a state desired by the surgeon.

### Modified Example 3

In the above-described exemplary embodiment, a case of displaying a medical image resembling actual appearance during surgery has been described. The exemplary embodiment is not limited to this, and the medical apparatus 40 may have various display modes for displaying medical images. For example, the medical apparatus 40 has a “real display mode”, an “attention alert mode”, and an “informed consent mode”, and displays a medical image suitable for each mode.

Here, the “real display mode” is a mode of displaying a medical image resembling actual appearance during surgery as described above. The “attention alert mode” is a mode that draws attention during surgery. The “informed consent mode” is a mode used when an explanation is provided to a subject. For example, the medical apparatus 40 may switch between these modes in accordance with a workflow regarding surgical treatment.

For example, in the workflow regarding surgical treatment, in a case where an explanation about a surgical treatment is to be provided to a subject, the change function 454 shifts a mode to the “informed consent mode” to change the display state of a medical image to an easily viewable state. For example, to improve the viewability of the target area, the change function 454 enhances the boarder of the target area in the medical image, shadows the target area, changes the direction of a shadow, and/or shadows the surface of the target area, thus changing the display state of a medical image. Thus, the change function 454 further changes the display state of a medical image in the explanation about surgical treatment to a subject.

For example, in the workflow regarding surgical treatment, in the case of performing treatment planning or surgery simulation, during surgery, the change function 454 shifts the mode to the “real display mode”, and executes the above-described processing.

For example, in a case where the target area is close to large blood vessels or nerves, the change function 454 shifts the mode to the “attention alert mode”, and further changes the display state of the medical image at the timing of attention alert during the procedure. For example, the change function 454 shifts the mode to the “attention alert mode”, so that in a case where the treatment section of the endoscope 31a has approaches blood vessels or nerves during endoscopic surgery, the change function 454 changes a display state in such a manner as to display the medical image in a color that draws attention. In this case, for example, the change function 454 acquires the position of the treatment section of the endoscope 31a that has been detected by the position sensor, monitors a distance between the acquired position of the treatment section and the positions of blood vessels or nerves in the above-described simulation model, and controls the display state of the medical image to be changed in a case where the distance falls below a threshold value.

### Modified Example 4

In addition to the above-described display control, the medical apparatus 40 can also perform display control based on the elapsed time of the procedure. Specifically, the change function 454 further changes the display state of the medical image based on the elapsed time since the start of the surgical treatment. For example, the change function 454 performs control to reduce blue light of a display that displays the medical image, based on the elapsed time since the start of the surgical treatment.

### Modified Example 5

In Modified Example 3 described above, the case of performing display for drawing attention in accordance with a distance between the treatment section of the endoscope 31a and blood vessels and/or nerves in the “attention alert mode” has been described. The exemplary embodiment is not limited to this, and the display state of a medical image may be changed in accordance with a distance between the treatment section and the target area, and/or the orientation of the treatment section (lens) with respect to the target area. For example, the change function 454 performs control to add reflected light to the medical image in such a manner as to represent the moisture on the surface of the target area.

For example, the change function 454 changes the display state of a medical image to brighten the surface of the target area and increase the intensity of the reflected light as the distance between the treatment section and the target area decreases. Additionally, the change function 454 changes the display state of the medical images so that the intensity of the reflected light from the target area increases as the orientation of the treatment section (lens) becomes more perpendicular to the surface of the target area, and decreases as the orientation of the treatment section (lens) becomes more oblique to the surface of the target area.

### Modified Example 6

In the above-described exemplary embodiment, a case of acquiring appearance information about the target area that is based on apparatus information about an apparatus used in surgery has been described. The exemplary embodiment is not limited to this, and appearance information about the target area that is based on a surgical instrument used in surgery may be acquired. In this case, for example, the acquisition function 452 reads out the above-described simulation model, illuminates the three-dimensional model of the surgical light 21 based on the illumination conditions at the present time, and acquires the appearance information about the target area that corresponds to a case where a surgical instrument is further arranged in a virtual space of a simulation model. Here, the type of the surgical instrument and the arrangement of the surgical instrument into the virtual space can be freely set by a manipulator (e.g., doctor, etc.). In other words, the manipulator can select a surgical instrument and arrange the surgical instrument at any position in the virtual space via the input interface 42. For example, the manipulator arranges a surgical instrument into the virtual space in accordance with a standing position of a doctor during surgery.

The acquisition function 452 reads information (information about the shape, size, etc.) about the surgical instrument that has been set by the manipulator, from the storage circuitry 44, and acquires the appearance information about the target area that corresponds to a case where the surgical instrument is arranged at a set position in the virtual space (information about a shadow caused by the surgical instrument, etc.). The storage circuitry 44 prestores information about a surgical instrument to be used in surgery (information about the shape and size of each type of surgical instrument).

The change function 454 changes the display state of a medical image in such a manner that appearance information that is based on the surgical instrument is reflected in the medical image. For example, the change function 454 changes the display state of a medical image in such a manner that a shadow formed by the surgical instrument is displayed within the medical image. With this configuration, for example, in displaying a simulation image of surgery, a change in the appearance of the target area that is based on the medical instrument used in the simulation can be displayed on the simulation image, and the medical image closer to actual appearance during surgery can be displayed.

As described above, according to the first exemplary embodiment, the acquisition function 452 acquires appearance information about the target area to be observed during a procedure in surgical treatment on a subject. The change function 454 changes, based on the appearance information, the display state of a captured medical image of the target area. Accordingly, the medical apparatus 40 according to the first exemplary embodiment can display a medical image suitable for the appearance of the target area, so that the display state of the target area included in the medical image resembles the appearance of the target area to be observed during surgical treatment.

Further, according to the first exemplary embodiment, the acquisition function 452 acquires appearance information about the target area that is based on the illumination conditions of a lighting apparatus to be used during a procedure in surgical treatment. The change function 454 changes the display state of a medical image in such a manner that the display state of the target area in the medical image resembles the appearance of the target area that is indicated by the appearance information. Accordingly, the medical apparatus 40 according to the first exemplary embodiment can display a medical image that is similar to the actual appearance of the target area and natural for a doctor.

Further, according to the first exemplary embodiment, the acquisition function 452 acquires appearance information about the target area in a captured image that is based on a display condition of a captured image of the target area that has been captured during a procedure of surgical treatment. The change function 454 changes the display state of a medical image in such a manner that the display state of the target area in the medical image resembles the appearance of the target area that is indicated by the appearance information. Accordingly, the medical apparatus 40 according to the first exemplary embodiment can display a medical image that is similar to the captured image and natural for a doctor.

Further, according to the first exemplary embodiment, the acquisition function 452 acquires appearance information about the target area that is based on the illumination conditions of a lighting apparatus to be used during a procedure. Accordingly, the medical apparatus 40 according to the first exemplary embodiment can acquire information regarding the actual appearance of the target area.

According to the first exemplary embodiment, the acquisition function 452 acquires appearance information about the target area that is based on the illumination conditions of a lighting apparatus in each progress status of a procedure. Each time appearance information is acquired, the change function 454 changes the display state of a medical image based on the acquired appearance information. Accordingly, the medical apparatus 40 according to the first exemplary embodiment can change the display state of the medical image in accordance with a change in appearance of the target area.

Further, according to the first exemplary embodiment, in a case where surgical treatment is performed through remote manipulation, the change function 454 changes the display state of a medical image in accordance with a manipulator of a remote manipulation who observes a captured image of the target area. Accordingly, the medical apparatus 40 according to the first exemplary embodiment can display a medical image desired by a doctor who executes telesurgery.

Further, according to the first exemplary embodiment, the change function 454 further changes the display state of a medical image at a timing of attention alert during a procedure. Accordingly, the medical apparatus 40 according to the first exemplary embodiment can support more safe execution of a procedure.

Further, according to the first exemplary embodiment, the change function 454 further changes the display state of a medical image in explanation about surgical treatment to a subject. Accordingly, the medical apparatus 40 according to the first exemplary embodiment can display a medical image that is easily viewable by a subject (patient).

Further, according to the first exemplary embodiment, the change function 454 further changes the display state of a medical image based on the elapsed time since the start of surgical treatment. Accordingly, the medical apparatus 40 according to the first exemplary embodiment can change the display state of a medical image in accordance with the state of a doctor who executes surgery.

Further, according to the first exemplary embodiment, the change function 454 changes, as the display state of a medical image, at least one of contrast and color. Accordingly, the medical apparatus 40 according to the first exemplary embodiment can appropriately change the display state of a medical image.

### Second Exemplary Embodiment

In the above-described first exemplary embodiment, the case of changing a display condition of a medical image based on appearance information about the target area that corresponds to the illumination conditions of a lighting apparatus or a captured image (endoscopic image) has been described. In the second exemplary embodiment, a case of changing the illumination conditions of a lighting apparatus or the display state of a captured image based on the display condition of a medical image will be described. A medical apparatus 40 according to the second exemplary embodiment differs from the first exemplary embodiment in the processing to be performed by the acquisition function 452 and the processing to be performed by the change function 454. Hereinafter, these points will be mainly described.

An acquisition function 452 according to the second exemplary embodiment acquires display conditions of a captured medical image of the target area of surgical treatment on a subject. Specifically, the acquisition function 452 acquires information regarding the contrast and the color of each portion that correspond to when a medical image such as a presurgical image, a treatment plan image, or a simulation image of a target person who is subjected to surgical treatment is displayed on a display.

In a case where a display condition of a medical image is changed, the acquisition function 452 also acquires the changed display condition. For example, the input interface 42 receives a change operation of changing a display condition of a medical image. The acquisition function 452 acquires the display conditions of the medical image that is set after the change operation.

A change function 454 according to the second exemplary embodiment changes the appearance of the target area to be observed during a procedure in surgical treatment, based on the display conditions of a medical image. Specifically, the change function 454 changes the appearance of the target area in such a manner that the appearance of the target area to be observed during a procedure in surgical treatment, resembles the display state of a medical image.

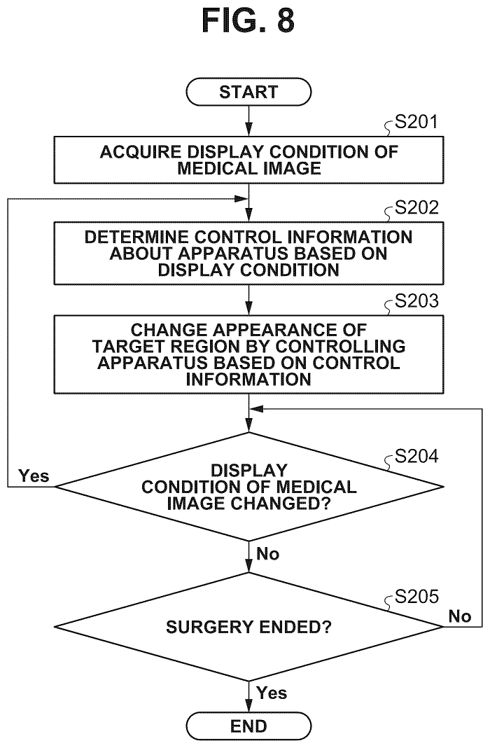

Next, a procedure of processing to be performed by the medical apparatus 40 according to the second exemplary embodiment will be described with reference to FIG. 8, and then details of each piece of processing will be described. FIG. 8 is a flowchart illustrating an example of a processing procedure of the medical apparatus 40 according to the second exemplary embodiment.

For example, in the present exemplary embodiment, the acquisition function 452 acquires, in step S201, display conditions of a medical image collected from a subject who is subjected to surgical treatment, as illustrated in FIG. 8. The processing is implemented by, for example, the processing circuitry 45 calling a program corresponding to the acquisition function 452, from the storage circuitry 44, and executing the program.

Subsequently, in step S202, the change function 454 determines control information about an apparatus based on the display conditions, and in step S203, changes the appearance of the target area by controlling the apparatus based on the control information. Specifically, the change function 454 transmits the determined control information to the input device 22 or the input device 32, thus controlling an apparatus in the surgery room 1 or the surgery room 2. The control information is transmitted by the control function 451. The processing is implemented by, for example, the processing circuitry 45 calling programs corresponding to the change function 454 and the control function 451, from the storage circuitry 44, and executing the programs.

In step S204, the change function 454 determines whether the display state of a medical image has been changed. The processing is implemented by, for example, the processing circuitry 45 calling a program corresponding to the change function 454, from the storage circuitry 44, and executing the program.

If it is determined in step S204 that the display state has been changed (YES in step S204), the processing returns to step S202, in which the change function 454 determines control information based on the display conditions after the change. If it is determined in step S204 that the display state has not been changed (NO in step S204), the processing proceeds to step S205. In step S205, the change function 454 determines whether the surgery has ended. If the surgery has not ended (NO in step S205), the processing returns to step S204. In step S204, the change function 454 continues the determination in step S204. If the surgery has ended (YES in step S205), the medical apparatus 40 ends the processing. The processing is implemented by, for example, the processing circuitry 45 calling a program corresponding to the change function 454, from the storage circuitry 44, and executing the program.

Hereinafter, details of each process to be performed by the medical apparatus 40 according to the second exemplary embodiment will be described. The medical apparatus 40 according to the second exemplary embodiment also executes a different type of processing between a case where celiotomy is performed and a case where endoscopic surgery is performed, as surgical treatment. Hereinafter, processing to be performed during celiotomy and processing to be performed during endoscopic surgery will be described in order.

### (Processing to be Performed During Celiotomy)

Hereinafter, processing to be performed during celiotomy will be described along the processing described with reference to the flowchart in FIG. 8.

### (Acquisition Processing of Display Condition)

As described in conjunction with step S201 of FIG. 8, the acquisition function 452 acquires the display conditions of a medical image collected from a subject on which surgical treatment is performed. For example, in surgical treatment, in the observation of a presurgical medical image or the observation of an intraoperative medical image, the display state of the medical image is sometimes changed to various states. For example, the orientation of light, the illuminance, and/or a wavelength in the medical image to be displayed is/are changed to improve the viewability of the target area depicted in the medical image. For example, a doctor who observes a medical image performs a change operation via the input interface 42, so that a display condition of a medical image is changed. The acquisition function 452 acquires such display conditions of a medical image each time a display condition is changed.

### (Determination Processing of Control Information)

As described with reference to step S202 of FIG. 8, the change function 454 determines control information for controlling an apparatus, based on the display conditions of the medical image that have been acquired in step S101. Specifically, the change function 454 initially acquires apparatus information corresponding to surgery information, from the apparatus information 441 stored in the storage circuitry 44. As in the first exemplary embodiment, the surgery information is acquired by the acquisition function 452. For example, the change function 454 acquires information (manufacturer name, model number, catalog information, etc.) regarding the surgical light 21 as apparatus information.

The change function 454 changes an illumination condition of a lighting apparatus to be used during a procedure in surgical treatment, in such a manner that the appearance of the target area to be observed during the procedure in surgical treatment resembles the display state of the medical image of which any display condition(s) has/have been changed with the change operation. The change function 454 determines control information in which the illumination conditions of the surgical light 21 are set in such a manner that the appearance of the target area becomes similar to the display state of the medical image.

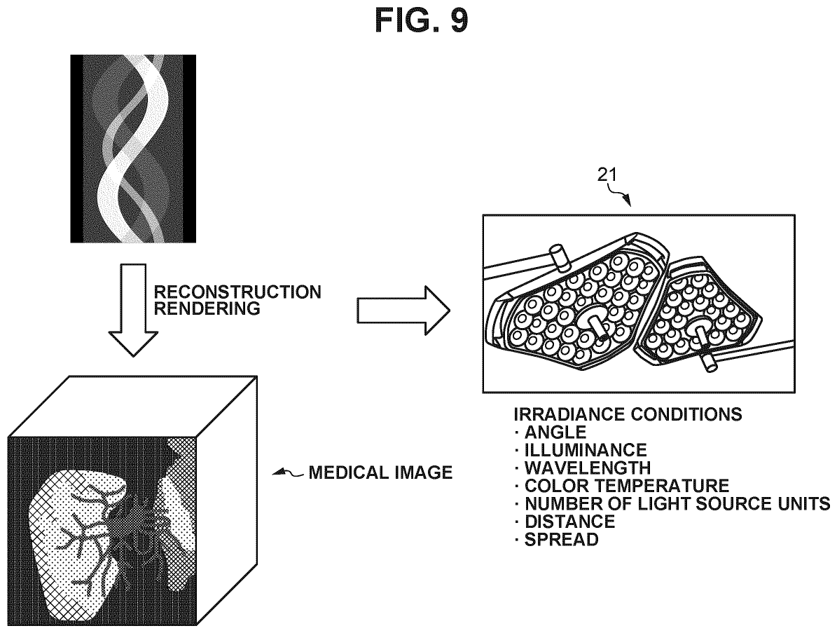

FIG. 9 is a diagram illustrating an example of apparatus control according to the second exemplary embodiment. For example, as illustrated in FIG. 9, the change function 454 determines the illumination condition(s) of the surgical light 21 (at least one of the angle of light, the illuminance of light, the wavelength of light, and the color temperature of light, the number of light source units, the distance from the respective light source units to the target area, and the spread of light) in such a manner that the appearance of the target area resembles the display state of the medical image, based on the display condition of the medical image generated through various types of reconstruction processing and rendering processing.

For example, the change function 454 determines control information using the above-described simulation model (simulation model in which a three-dimensional model of a surgical table, a three-dimensional model of the surgical light 21, and a three-dimensional model of a subject are arranged in a virtual space simulating the surgery room 1). In this case, the change function 454 determines control information for controlling the actual surgical light 21, by determining the position and the orientation of the surgical light 21, an illuminance and a wavelength of light emitted from each light source in the simulation model, based on the display conditions of the medical image (orientation, illuminance, and wavelength of light to the target area, etc.).

By simulating he appearance of the target area illuminated under the illumination condition of the surgical light 21 that has been determined as described above, using the above-described simulation model, and comparing a simulation result and the display state of a medical image, it is possible to check the accuracy of control information. In this case, for example, the change function 454 may calculate a similarity degree between the appearance of the target area (contrast and color of each portion, etc.) obtained through simulation, and the display state of the medical image (contrast and color of each portion, etc.), and determine an illumination condition corresponding to a case where the similarity degree exceeds a threshold value, as control information.

Each time a display condition is acquired by the acquisition function 452, the change function 454 executes the above-described processing, and determines control information for controlling the surgical light 21. For example, at the time of presurgical surgery planning, a doctor adjusts illumination while referencing a medical image, so that conditions such as an angle and an illuminance at which the target area does not become less visible by light reflection are set. The change function 454 controls the surgical light 21 based on the set condition. For example, at the time of presurgical surgery planning, a doctor adjusts the size of a surgical field while referencing a medical image. The change function 454 determines the illumination conditions of the surgical light 21 in such a manner that light is emitted to the set surgical field, and controls the surgical light 21.

### (Apparatus Control Processing)

As described in conjunction with step S203 of FIG. 8, the control function 451 transmits control information to the input device 22, so that the change function 454 changes the illumination conditions of the surgical light 21. With this configuration, the appearance of the target area during celiotomy is changed to resemble the display state of a medical image. The apparatus control may be automatically performed through transmission of control information to the input device 22 as described above. Alternatively, the apparatus control may be performed by control information being displayed on the display 23 and the manipulator manipulating the surgical light 21 based on the displayed control information.

### (Change Determination Processing)

As described in conjunction with step S204 of FIG. 8, the change function 454 determines whether a display condition of a medical image has been changed. For example, at the time of observation of a presurgical medical image or observation of an intraoperative medical image, the display state of the medical image is sometimes changed (e.g., a view angle or an area desired to be viewed is changed). If a change in display condition is detected by the change function 454, the acquisition function 452 acquires the display conditions after the change. Each time a display condition is acquired, the change function 454 determines control information about the surgical light 21.

### (Processing End Determination Processing)

As described in conjunction with step S205 of FIG. 8, the change function 454 determines whether the surgery has ended. For example, the change function 454 determines whether the surgery has ended, by determining whether the display of the medical image on the display 23 has been ended.

In above-described exemplary embodiments, a case of determining control information in accordance with the display conditions of a medical image has been described. The exemplary embodiment is not limited to this. For example, control information may be changed in accordance with the number or the standing position of a doctor who performs surgery. For example, based on the standing position of each doctor, control information about the surgical light 21 is determined in such a manner that no shadow is formed on the target area. In this case, the standing position of each doctor is predetermined, a three-dimensional model of each doctor is arranged at the corresponding position in the above-described simulation model, for example, and control information about the surgical light 21 is determined.

### (Processing to be Performed During Endoscopic Surgery)

Next, processing to be performed during endoscopic surgery will be described along the processing described with reference to the flowchart in FIG. 8.

### (Acquisition Processing of Display Condition)

In the processing to be performed during endoscopic surgery, the acquisition function 452 also acquires the display conditions of a medical image, as in celiotomy.

### (Determination Processing of Control Information)

In the processing to be performed during endoscopic surgery, the change function 454 acquires, as apparatus information corresponding to surgery information, information (manufacturer name, model number, catalog information, etc.) regarding the endoscope system 31. The change function 454 changes the display conditions of a captured image in such a manner that the appearance of the target area in a captured image captured during a procedure in surgical treatment resembles the display state of a medical image of which the display condition(s) has/have been changed through a change operation. In other words, the change function 454 determines control information in which the conditions of the endoscope system 31 are set in such a manner that the display state of an endoscopic image becomes similar to the display state of the medical image.

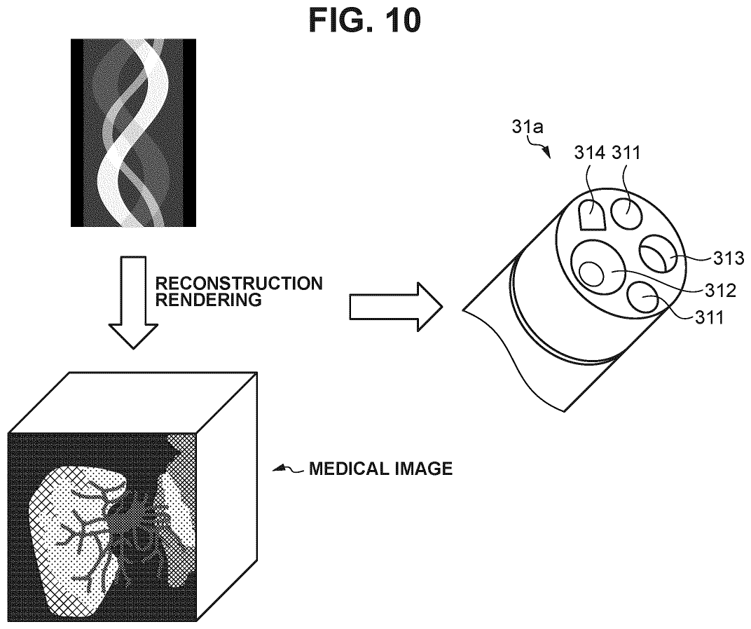

FIG. 10 is a diagram illustrating an example of apparatus control according to the second exemplary embodiment. For example, as illustrated in FIG. 10, the change function 454 determines the conditions of the endoscope 31a in such a manner that the display state of an endoscopic image resembles the display state of a medical image generated through various types of reconstruction processing and rendering processing, based on the display conditions of the medical image. As illustrated in FIG. 10, the endoscope 31a includes, for example, illumination lenses 311, an objective lens 312, a treatment section channel 313, and a lens cleaning nozzle 314 that are provided at the leading end.

Each illumination lens 311 is a light emission unit that emits light emitted from an internal light source. As illustrated in FIG. 10, for example, two illumination lenses 311 are provided at the leading end of the endoscope 31a. The objective lens 312 is a lens for image capturing that includes therein an image sensor. The treatment section channel 313 accommodates therein a medical instrument such as forceps, and is configured to output the medical instrument to the outside at the time of endoscopic surgery. The lens cleaning nozzle 314 ejects cleaning water for cleaning the illumination lenses 311 and the objective lens 312.

For example, the change function 454 determines control information using the above-described simulation model (simulation model in which a three-dimensional model of a surgical table, a three-dimensional model of the endoscope 31a, and a three-dimensional model of a subject are arranged in a virtual space simulating the surgery room 2). In this case, the change function 454 determines control information for controlling the actual endoscope 31a, by determining how light is emitted from the illumination lenses 311 of the endoscope 31a in the simulation model (emits light from both illumination lenses 311, or emits light from either one of them, etc.), and the illuminance and the wavelength of light emitted from the respective illumination lenses 311, based on the display conditions of a medical image (the orientation, illuminance, and wavelength of light to the target area, etc.).

In a case where the endoscope system 31 performs a procedure using a surgery support robot, the change function 454 can also determine control information for controlling the orientation of light to be emitted, and the position of the endoscope 31a with respect to the target area.

By comparing an endoscopic image captured by the endoscope 31a controlled based on control information determined as described above, and the display state of a medical image, it is possible to also check the accuracy of control information. In this case, the change function 454 may calculate a similarity degree between the display state of the endoscopic image (contrast and/or color of each portion, etc.), and the display state of the medical image (contrast and/or color of each portion, etc.), for example, and determine a condition in a case where the similarity degree exceeds a threshold value to be control information.

Each time the display conditions are acquired by the acquisition function 452, the change function 454 executes the above-described processing, and determines control information for controlling the endoscope 31a.

### (Apparatus Control Processing)

In the processing to be performed during endoscopic surgery, the control function 451 transmits control information to the input device 32, so that the change function 454 changes a control condition of an endoscope system. With this configuration, the display state of an endoscopic image is changed to resemble the display state of a medical image. The apparatus control may be automatically performed by transmitting control information to the input device 32 as described above. Alternatively, the apparatus control may be performed by displaying control information on the display 31b and the manipulator manipulating the endoscope system 31 based on the displayed control information.

### (Change Determination Processing)

In the processing to be performed during endoscopic surgery, the change function 454 determines whether a display condition of a medical image has been changed, as in celiotomy.

### (Processing End Determination Processing)

In the processing to be performed during endoscopic surgery, the change function 454 determines whether surgery has ended, by determining whether the display of the medical image on the display 31b has been ended, for example.

In the above-described exemplary embodiment, a case of determining control information in accordance with the display conditions of a medical image has been described. The exemplary embodiment is not limited to this. For example, in a case where surgery is performed by a surgery support robot, control information may be changed in accordance with the position of a robot arm. For example, based on the position of the robot arm, control information about the endoscope 31a is determined in such a manner that a shadow is not formed on the target area. In this case, the position of the robot arm is acquired, a three-dimensional model of the robot arm is arranged at the corresponding position in the above-described simulation model, for example, and the control information about the endoscope 31a is determined.

In the above-described exemplary embodiment, the case to be applied to surgery in a hospital illustrated in FIG. 1 has been described. The exemplary embodiment is not limited to this. The case may be applied to telesurgery as illustrated in FIG. 7. In this case, a surgery support robot and/or an endoscope to be operated by the surgery support robot are controlled in such a manner that an endoscopic image displayed on a display of a console for operating a surgery support robot present at a remote location enters a display state resembling a medical image.

### Modified Example 1

In the above-described exemplary embodiment, a case of changing the display state of an endoscopic image by changing conditions in image capturing has been described. The exemplary embodiments are not limited to this. For example, the display state of an endoscopic image may be changed by changing a display mode in displaying an endoscopic image on a display.

For example, in a case where an organ or an area that is desired to be distinguished from the others is present, in order to display these in a distinguishable manner, the change function 454 changes a display mode of an endoscopic image by enhancing a boarder within the endoscopic image, shadowing the area, changing the direction of a shadow, and/or shading the surface of the area.

### Modified Example 2

In the above-described exemplary embodiments, a case where the lighting conditions of the surgical light 21 and the control conditions of the endoscope 31a are changed in accordance with a change in the display state of a medical image has been described. The exemplary embodiments are not limited to this. For example, control may be performed at a timing desired by a doctor. In this case, for example, if a switch button is pressed by a doctor, the change function 454 controls the lighting conditions of the surgical light 21 and the control conditions of the endoscope 31a to change.

### Modified Example 3

In the medical apparatus 40 according to the second exemplary embodiment, also the “attention alert mode” can be included, as in the first exemplary embodiment. In this case, the change function 454 further changes display condition(s) of a captured image at the timing of attention alert during a procedure. For example, the change function 454 shifts the mode to the “attention alert mode”, so that in a case where the treatment section of the endoscope 31a approaches blood vessels or nerves during endoscopic surgery, the change function 454 changes the display state in such a manner as to display an endoscopic image in a color that draws attention. In this case, for example, the change function 454 acquires the position of the treatment section of the endoscope 31a that has been detected by the position sensor, monitors the distance between the acquired position of the treatment section and the positions of blood vessels or nerves in the above-described simulation model, and controls the display state of an endoscopic image to be changed in a case where the distance falls below a threshold value.

### Modified Example 4

In the medical apparatus 40 according to the second exemplary embodiment, it is possible to perform control suitable for an elapsed time in a procedure, as in the first exemplary embodiment. Specifically, the change function 454 further changes illumination conditions of a lighting apparatus in accordance with an elapsed time since the start of the surgical treatment. For example, in a case where the surgical light 21 is configured to change the intensity of red, blue, and green, the change function 454 can perform control of the color(s) in accordance with an elapsed time of surgery. For example, the change function 454 controls blue-color light emitted from the surgical light 21, to gradually decrease in accordance with an elapsed time since the start of the surgical treatment. Since blue-color light causes eye fatigue, this control reduces eye fatigue.

Additionally, long-time viewing of red color causes eye fatigue. For this reason, the change function 454 controls red-color light emitted from the surgical light 21, to gradually reduce as a total bleeding amount during surgery increases. For example, the change function 454 estimates a bleeding amount during celiotomy, based on an image from a camera installed in the surgery room 1. The change function 454 controls red-color light emitted from the surgical light 21, to decrease as a total bleeding amount increases. This control reduces eye fatigue and optic illusion.

Here, the change function 454 can also control the intensity of red-color light to return to the original intensity if a sudden large amount of bleeding occurs. This makes the field of view appear red, providing a visual warning of significant bleeding.

### Modified Example 5

In the above-described exemplary embodiment, a case where lighting conditions of the surgical light 21 and/or control conditions of the endoscope 31a are changed in accordance with a change in the display state of a medical image has been described. The medical apparatus 40 can also output information that is based on the display state of the medical image. Specifically, the medical apparatus 40 can output support information that is based on simulation performed using the display state of the medical image.

For example, in a case where the lighting conditions of the surgical light 21 are determined based on the display state of a medical image, the medical apparatus 40 can output the standing positions of doctors at which no shadow is formed, as support information. In this case, when the lighting conditions of the surgical light 21 are determined using the above-described simulation model, the change function 454 determines whether a shadow is formed on the target area in a case where three-dimensional models of the doctors are each arranged at the corresponding position in the virtual space of the simulation model. The change function 454 identifies a position in the virtual space where no shadow is formed on the target area even in a case where the three-dimensional models of the doctors are arranged. The control function 451 displays a position in the surgery room 1 that corresponds to the position identified by the change function 454, on the display 43 or the display 23 as support information. By viewing the information, the doctors can easily recognize a position where no shadow is formed.

For example, in the case of determining a control condition of the endoscope 31a based on the display state of a medical image, the medical apparatus 40 can output an insertion position of the insertion section as support information. For example, in the case of determining the control conditions of the endoscope 31a using the above-described simulation model, the change function 454 determines the insertion position of the insertion section within a subject on the three-dimensional model where no shadow is formed. The control function 451 displays a position on the three-dimensional model that has been identified by the change function 454, on the display 43 and/or the display 23 as support information. By viewing the information, a doctor can easily recognize the insertion position of the insertion section where no shadow is formed.

As described above, according to the second exemplary embodiment, the acquisition function 452 acquires the display condition of a captured medical image of the target area of surgical treatment on a subject. The change function 454 changes the appearance of the target area to be observed during the procedure in surgical treatment, based on the display conditions of the medical image. Accordingly, the medical apparatus 40 according to the second exemplary embodiment can show the appearance of the target area that corresponds to the display state of a medical image, and can cause the display state of the target area included in the medical image, to resemble the appearance of the target area to be observed during surgical treatment.

According to the second exemplary embodiment, the input interface 42 receives a change operation of changing a display condition of a medical image. The acquisition function 452 acquires the display conditions of the medical image that have been set after the change operation. The change function 454 changes an illumination condition of a lighting apparatus to be used during a procedure in surgical treatment, in such a manner that the appearance of the target area to be observed during the procedure in surgical treatment, resembles the display state of the medical image of which the display condition(s) has/have been changed through a change operation. Accordingly, the medical apparatus 40 according to the second exemplary embodiment can observe the target area with the appearance resembling the display state when the medical image is observed.

According to the second exemplary embodiment, the input interface 42 receives a change operation of changing display condition(s) of a medical image. The acquisition function 452 acquires the display conditions of the medical image that are set after the change operation. The change function 454 changes the display conditions of a captured image captured during a procedure in surgical treatment in such a manner that the appearance of the target area in the captured image, resembles the display state of the medical image of which the display condition(s) has/have been changed through a change operation. Accordingly, the medical apparatus 40 according to the second exemplary embodiment can observe a captured image resembling the display state corresponding to when the medical image is observed.

According to the second exemplary embodiment, the change function 454 further changes the display state of a captured image at a timing of attention alert during a procedure. Accordingly, the medical apparatus 40 according to the second exemplary embodiment can support more safe execution of a procedure.

According to the second exemplary embodiment, the change function 454 further changes illumination condition(s) of a lighting apparatus in accordance with an elapsed time since the start of the surgical treatment. Accordingly, the medical apparatus 40 according to the second exemplary embodiment can change a lighting environment in accordance with the state of a doctor who executes surgery.

According to the second exemplary embodiment, the change function 454 changes, as illumination condition(s) of a lighting apparatus, at least one of the angle of illumination, the illuminance of illumination, the wavelength of illumination, and the color temperature of illumination, the number of light source units, the distance from each light source unit to the target area, and light spread from each light source unit. Accordingly, the medical apparatus 40 according to the second exemplary embodiment can appropriately change light emitted from the lighting apparatus.

### Third Exemplary Embodiment

In the third exemplary embodiment, variations of display of a medical image and a captured image (e.g., endoscopic image) will be described. A medical apparatus 40 according to the third exemplary embodiment differs from those in the first exemplary embodiment and the second exemplary embodiment in processing performed by the control function 451. Hereinafter, this point will mainly be described.

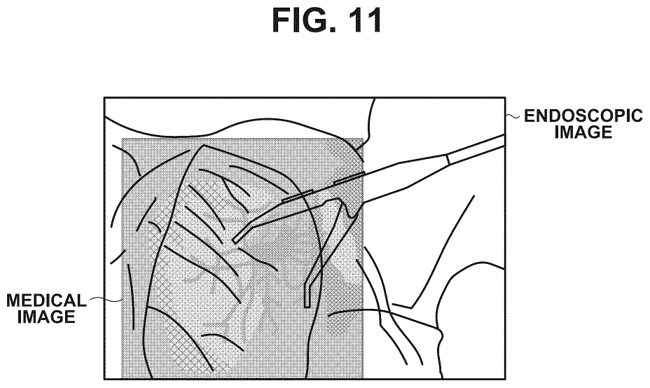

A control function 451 according to the third exemplary embodiment displays a medical image and a captured image in a superimposed manner. FIG. 11 is a diagram illustrating an example of a display image according to the third exemplary embodiment. For example, as illustrated in FIG. 11, the control function 451 can display a display image in which a medical image and an endoscopic image are superimposed on the display 43 and the display 31b.

The control function 451 displays a display image in which a medical image and an endoscopic image are superimposed with a condition of contrast and a condition of color in the respective images being matched. As described in the first exemplary embodiment or the second exemplary embodiment, the control function 451 displays the display image in which images with the display state of the medical image and the display state of the captured image (endoscopic image) that are similar to each other, are superimposed.

The control function 451 can also further change the display state of each image in such a manner that light reflection reduces in each image, and display a display image in which these images are superimposed.

The control function 451 can also display a display image in which images (medical image and captured image) with the display states that have not been made similar are superimposed. For example, the control function 451 can also display a captured image including light reflection, and a medical image not including light reflection, in a superimposed manner. This enables, for example, a less visible portion in the captured image due to light reflection to be checked in the medical image.

As described above, according to the third exemplary embodiment, the control function 451 displays a medical image and a captured image in a superimposed manner. Accordingly, the medical apparatus 40 according to the third exemplary embodiment enables the display of easily observable superimposed images.

### Other Exemplary Embodiments

In above-described exemplary embodiments, a case of changing the display state of a medical image in such a manner as to resemble the appearance of the target area (contrast, color, and shadow states of each portion) has been described. The exemplary embodiments are not limited to this. For example, the display state of the medical image may be changed in accordance with a progress status of the surgery. In this case, for example, the acquisition function 452 acquires information regarding a progress status of celiotomy. As an example, the acquisition function 452 acquires an image captured by a camera installed in the surgery room 1. By analyzing the acquired image, the acquisition function 452 acquires information regarding a progress status of surgery (e.g., whether the abdominal opening has been made).

In a case where the progress status acquired by the acquisition function 452 indicates that the surgery is before the abdominal opening, the change function 454 performs control to cause the generation function 453 to perform generation through surface rendering to generate a medical image representing a body surface. In a case where the progress status acquired by the acquisition function 452 indicates that the surgery is after the abdominal opening, the change function 454 controls the generation function 453 to generate a medical image showing structures such as blood vessels by using volume rendering and adjusting the transparency.

In the above-described exemplary embodiment, an example case where a control unit, acquisition unit, generation unit, and a change unit in this specification are respectively implemented by a control function, an acquisition function, a generation function, and a change function of the processing circuitry has been described, but the exemplary embodiments are not limited to these. For example, the control unit, the acquisition unit, the generation unit, and the change unit in this specification can be implemented by not only the control function, the acquisition function, the generation function, and the change function described in the exemplary embodiments, but also by hardware alone, software alone, or a combination of hardware and software.

The term “processor” used in the description of the foregoing exemplary embodiments refers to a circuit such as a central processing unit (CPU), a graphics processing unit (GPU), an application specific integrated circuit (ASIC), and a programmable logic device (e.g., a simple programmable logic device [SPLD], a complex programmable logic device [CPLD], or a field programmable gate array [FPGA]). Instead of storing the programs in the storage circuit, the programs may be directly built in the processor circuit. In such a case, the processor implements the functions by reading the built-in programs in its own circuit and executing the programs. The processors according to the exemplary embodiments are not limited to a single-circuit configuration. A plurality of independent circuits may be combined into a processor that implements the functions.

A control program to be performed by a processor includes being preliminarily installed on a ROM or storage circuitry. This control program may be provided with being recorded on a non-transitory computer-readable storage medium such as a compact disk (CD)-ROM, a flexible disk (FD), a CD-recordable (R), or a digital versatile disk (DVD) in a file with a format installable into or executable on these apparatuses. This control program may be provided or distributed by being stored on a computer connected to a network such as the internet, and downloaded via the network. For example, this control program includes modules having the above-described each processing functions. As actual hardware, by a central processing unit (CPU) reading out a medical image processing program from a storage medium such as a ROM, and executing the medical image processing program, each module is loaded on a main storage device, and generated on the main storage device.

In the foregoing exemplary embodiments and modifications, the components of the apparatuses illustrated in the diagrams are functional concepts and do not necessarily need to be physically configured as illustrated in the diagrams. In other words, the specific forms of distribution or integration of the apparatuses are not limited to the illustrated ones, and all or part of the apparatuses can be functionally or physically distributed or integrated into any units depending on various loads and usages. All or part of the processing functions for the apparatuses to perform can be implemented by a CPU and programs to be analyzed and executed by the CPU, or as wired logic hardware.

All or part of the processes described to be automatically performed in the foregoing exemplary embodiments and modifications can be manually performed. All or part of the processes described to be manually performed can be automatically performed using known methods. Furthermore, the processing procedures, control procedures, specific names, and information including various types of data and parameters described above or illustrated in the drawings can be freely modified unless otherwise specified.

According to at least one of the exemplary embodiments described above, it is possible to cause the display state of a target area included in a medical image, to resemble the appearance of the target area to be observed during surgical treatment.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical apparatus, comprising processing circuitry configured to:
acquire appearance information about a target area to be observed during a procedure in surgical treatment on a subject; and
change a display state of a captured medical image of the target area based on the appearance information.

2. The medical apparatus according to claim 1,
wherein the processing circuitry acquires appearance information about the target area that is based on an illumination condition of a lighting apparatus to be used during the procedure in the surgical treatment, and
wherein the processing circuitry changes the display state of the medical image in such a manner that a display state of the target area in the medical image resembles appearance of the target area that is indicated by the appearance information.

3. The medical apparatus according to claim 1,
wherein the processing circuitry acquires appearance information about the target area in a captured image of the target area captured during the procedure of the surgical treatment, the appearance information being based on a display condition of the captured image, and
wherein the processing circuitry changes the display state of the medical image in such a manner that the display state of the target area in the medical image resembles appearance of the target area that is indicated by the appearance information.

4. The medical apparatus according to claim 2, wherein the processing circuitry acquires appearance information about the target area that is based on an illumination condition of a lighting apparatus that is being used during the procedure.

5. The medical apparatus according to claim 2,
wherein the processing circuitry acquires appearance information about the target area that is based on an illumination condition of the lighting apparatus for each progress status of the procedure, and
wherein, each time the appearance information is acquired, the processing circuitry changes the display state of the medical image based on the acquired appearance information.

6. The medical apparatus according to claim 3, wherein, in a case where the surgical treatment is performed through a remote manipulation, the processing circuitry changes the display state of the medical image in accordance with a manipulator of the remote manipulation who observes the captured image of the target area.

7. The medical apparatus according to claim 1, wherein the processing circuitry further changes the display state of the medical image at a timing of attention alert during the procedure.

8. The medical apparatus according to claim 1, wherein the processing circuitry further changes the display state of the medical image in an explanation about the surgical treatment to the subject.

9. The medical apparatus according to claim 1, wherein the processing circuitry further changes the display state of the medical image in accordance with an elapsed time since a start of the surgical treatment.

10. The medical apparatus according to claim 3, wherein the processing circuitry is further configured to display the medical image and the captured image in a superimposed manner.

11. The medical apparatus according to claim 1, wherein the processing circuitry changes at least one of contrast and color as the display state of the medical image.

12. A medical apparatus, comprising processing circuitry configured to:
acquire a display condition of a captured medical image of a target area of surgical treatment on a subject; and
change appearance of a target area to be observed during a procedure in the surgical treatment, based on the display condition of the medical image.

13. The medical apparatus according to claim 12, wherein the processing circuitry is further configured to receive a change operation of changing the display condition of the medical image,
wherein the processing circuitry acquires the display condition of the medical image that is set after the change operation, and
wherein the processing circuitry changes an illumination condition of a lighting apparatus to be used during the procedure in the surgical treatment, in such a manner that the appearance of the target area to be observed during the procedure in the surgical treatment resembles a display state of the medical image of which the display condition has been changed through the change operation.

14. The medical apparatus according to claim 12, wherein the processing circuitry is further configured to receive a change operation of changing the display condition of the medical image,
wherein the processing circuitry acquires the display condition of the medical image that is set after the change operation, and
wherein the processing circuitry changes a display condition of a captured image captured during the procedure in the surgical treatment, in such a manner that the appearance of the target area in the captured image resembles the display state of the medical image of which the display condition has been changed through the change operation.

15. The medical apparatus according to claim 14, wherein the processing circuitry further changes the display condition of the captured image at a timing of attention alert during the procedure.

16. The medical apparatus according to claim 13, wherein the processing circuitry further changes the illumination condition of the lighting apparatus in accordance with an elapsed time since a start of the surgical treatment.

17. The medical apparatus according to claim 14, wherein the processing circuitry is further configured to display the medical image and the captured image in a superimposed manner.

18. The medical apparatus according to claim 13, wherein the processing circuitry changes, as the illumination condition of the lighting apparatus, at least one of an angle of illumination, illuminance of illumination, a wavelength of illumination, a color temperature of illumination, the number of light source units, a distance, and spread.

19. A control method, comprising:
acquiring appearance information about a target area to be observed during a procedure in surgical treatment on a subject; and
changing a display state of a captured medical image of the target area based on the appearance information.

20. A control method, comprising:
acquiring a display condition of a captured medical image of a target area of surgical treatment on a subject; and
changing appearance of a target area to be observed during a procedure in the surgical treatment, based on the display condition of the medical image.
